(19) United States
(12) Patent Application Publication
(10) Pub. No.: US 2023/0026024 A1
(43) Pub. Date: Jan. 26, 2023

(54) SYSTEM AND METHOD FOR TWO-STEP SIMULTANEOUS CONVERSION OF CARBON DIOXIDE (CO2) AND HYDROCARBON CONTAINING AT LEAST ONE HYDROXY GROUP

(71) Applicant: KOREA RESEARCH INSTITUTE OF CHEMICAL TECHNOLOGY, Daejeon (KR)

(72) Inventors: Young Kyu HWANG, Daejeon (KR); Changho YOO, Daejeon (KR); Dong Won HWANG, Daejeon (KR); Do-Young HONG, Daejeon (KR); Jaedeuk PARK, Daejeon (KR); In Hyoup SONG, Daejeon (KR); Maeum LEE, Daejeon (KR); Kyung-Ryul OH, Daejeon (KR); Anil Haribhau VALEKAR, Daejeon (KR)

(73) Assignee: KOREA RESEARCH INSTITUTE OF CHEMICAL TECHNOLOGY, Daejeon (KR)

(21) Appl. No.: 17/858,528

(22) Filed: Jul. 6, 2022

(30) Foreign Application Priority Data
Jul. 9, 2021 (KR) 10-2021-0090481

(51) Int. Cl. C07C 51/41; C07C 51/44; C07C 51/15

(52) U.S. Cl. CPC C07C 51/41; C07C 51/44; C07C 51/15

(57) ABSTRACT

Proposed is a two-pot-two-step simultaneous conversion system for carbon dioxide and a hydrocarbon containing at least one hydroxy group and a method thereof. The system integrates a process of producing a metal salt that is a dehydrogenated form of the hydrocarbon, hydrogen, and water by reacting a hydrocarbon containing at least one hydroxy group in the presence of water and a metal oxalate, and a process of converting carbon dioxide or carbon dioxide-derived carbonate into formate by hydrogenation, thereby increasing energy efficiency while maintaining a higher hydrocarbon conversion rate and a higher carbon dioxide conversion rate than the one-pot conversion system for hydrocarbon and carbon dioxide.

The present application claims priority to Korean Patent Application No. 10-2021-0090481, filed Jul. 9, 2021, the entire contents of which is incorporated herein for all purposes by this reference.

### BACKGROUND OF THE INVENTION

### 1. Field of the Invention

The present disclosure relates to a two-step simultaneous conversion system for carbon dioxide and a hydrocarbon containing at least one hydroxy group and to a method thereof. More particularly, the present disclosure relates to a two-pot-two-step simultaneous conversion system for conversion of carbon dioxide and a hydrocarbon including at least one hydroxy group by integrating a process of producing a metal salt that is a dehydrogenated form of the hydrocarbon, hydrogen, and water by reacting a hydrocarbon containing at least one hydroxy group in the presence of water and a metal oxalate, and a process of converting carbon dioxide or carbon dioxide-derived carbonate into formate by hydrogenation, thereby increasing energy efficiency while maintaining a higher hydrocarbon conversion rate and a higher carbon dioxide conversion rate than the one-pot conversion system for hydrocarbon and carbon dioxide.

### 2. Description of the Related Art

Carbon dioxide (CO₂) is the most important substance among greenhouse gases specified in the Kyoto Protocol to the United Nations Framework Convention on Climate Change. Accordingly, countries around the world are making considerable efforts to develop technologies to control and reduce carbon dioxide emissions, such as increasing the efficiency of fossil energy use, developing low-carbon energy sources and alternative energy sources. However, the development and application of carbon dioxide disposal technology are essential in order to satisfy the emission levels suggested by the Kyoto Protocol. Accordingly, efforts have been made to capture carbon dioxide and convert it into other useful compounds.

Carbon capture and sequestration (CCS), which is currently recognized as a method for processing carbon dioxide, has a growing need for post-treatment technology due to environmental controversy, and as one of the solutions, carbon capture and utilization (CCU) is emerging.

Carbon dioxide capture, utilization, and storage technology is a technology field that uses carbon dioxide from industrial processes and power plants to produce valuable products and uses technology to store carbon dioxide. Carbon dioxide is a carbon compound having very low energy, and a large amount of energy is required to convert it into a useful resource, which hinders the commercial success of carbon dioxide conversion technology. Therefore, if a catalyst is developed to minimize energy use and improve the selectivity of the reaction, carbon dioxide conversion technology can be a very useful technology because added value is produced by converting carbon dioxide into a useful substance at a low cost.

One of the carbon dioxide conversion processes is a process for converting carbon dioxide into formic acid through a hydrogenation reaction and may be specifically represented by reaction formula 1 below. The formic acid is used in various industrial fields, such as livestock feed processing, leather dyeing, rubber synthesis. It is in the spotlight as a hydrogen storage material because of its low flammability and easy storage and transport.

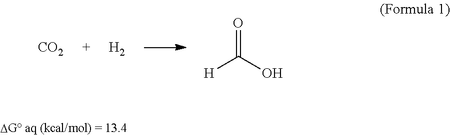

The hydrogenation reaction is widely used in the field of catalysts, but hydrogen gas is dangerous to transport and difficult to use, so instead of researching the hydrogenation reaction using hydrogen gas, research on converting carbon dioxide into formate by transferring hydrogen from the hydroxy group of a hydrocarbon containing at least one hydroxy group to carbon dioxide is in the spotlight. (Non-Patent Document 1).

As a representative example of the hydrocarbon containing at least one hydroxy group, glycerol is mentioned. The glycerol is produced as a by-product in the production process of biodiesel and is currently being simply incinerated as waste. In this process, a large amount of cost is required, and thus, the economy of the biodiesel production process is degraded, and a large amount of carbon dioxide is produced during the by-product incineration process, causing secondary environmental pollution. Accordingly, research on a method of utilizing glycerol, which is produced as a by-product in the biodiesel production process, is being conducted. If carbon dioxide is converted from the reaction of carbon dioxide and glycerol to formic acid and lactic acid, it can be a good alternative to solve both of glycerol treatment problem and the carbon dioxide conversion problem at the same time.

However, the reaction for converting hydrocarbon containing at least one hydroxy group such as glycerol into a metal salt that is a dehydrogenated form of the hydrocarbon by converting hydrocarbon in the presence of water and metal oxalates is performed at a relatively high temperature. Since the conversion reaction of carbon dioxide is performed at a relatively low temperature when hydrocarbon containing at least one hydroxyl group and carbon dioxide are simultaneously converted in one-pot, a reaction temperature suitable for each reaction is different and thus may not be reacted at the same temperature. Therefore, there is a problem in that the yield may be lowered or by-products may be produced by a high-temperature reaction, and the conversion product of a hydrocarbon and the conversion product of carbon dioxide may be mixed and separated.

According to the above circumstances, in the present disclosure, in the simultaneous conversion system of a hydrocarbon containing at least one hydroxyl group and carbon dioxide, the carbon dioxide and a hydrocarbon containing at least one hydroxy group were not reacted in the same place but were reacted through a separate route, and the hydrogen obtained from the dehydrogenation of the hydrocarbon was adjusted so that it could be used directly in the carbon dioxide conversion process. Therefore, the present disclosure relates to a two-step simultaneous conversion process of a hydrocarbon, including at least one hydroxy group and carbon dioxide, in which the process energy efficiency and convenience were improved by removing the process of producing/separating hydrogen for hydrogenation of carbon dioxide from the production site and transferring hydrogen by re-compression.

On the other hand, as related art in the same technical field as the present disclosure, U.S. Patent Publication No. US 2015-0299082 A1 (2015.10.22. Publication date) discloses a method for simultaneously producing lactic acid and propylene glycol from glycerol and discloses a process and apparatus for dehydrogenating a hydrocarbon containing at least one hydroxyl group using a dehydrogenation catalyst. In addition, US Patent Publication No. US 2016-0137573 A1 (2016.05.19. Publication date) relates to a catalyst system for producing formate, formic acid, or a mixture thereof from carbon dioxide and discloses a technology for producing formic acid by catalyzing bicarbonate and hydrogen gas. In addition, Korean Patent Publication No. KR 10-2020-0079035 A (2020.07.02. Publication date) discloses a method for producing a formate compound and a lactic acid compound through a hydrogenation conversion reaction of a carbonate compound using a hydrocarbon containing at least one hydroxy group.

The related art documents disclose a technology for dehydrogenating a hydrocarbon containing at least one hydroxy group to convert it to lactate, a technology for producing formic acid by catalytic reaction of bicarbonates and hydrogen gas, and for producing formic acid by catalytic reaction of a hydrocarbon containing at least one hydroxy group. However, the present disclosure may use a process of converting hydrocarbon containing at least one hydroxyl group and a process of producing carbon dioxide in a separate place, and hydrogen produced in the process of converting hydrocarbon containing at least one hydroxyl group may be used immediately in the process of producing carbon dioxide-derived carbonates. In addition, since the production yield of each process is improved and the process efficiency is increased, there is a difference between these related arts.

### DESCRIPTION OF THE RELATED ART

### Patent Literatures

(Patent literature 1) US Patent Application Laid-open

Publication No. US 2015-0299082 A1 (2015.10.22. publication date)

(Patent literature 2) US Patent Application Laid-open Publication No. US 2016-0137573 A1 (2016.05.19. publication date)

(Patent literature 3) Korean Patent Application Laid-open Publication No. KR 10-2020-0079035 A (2020.07.02. publication date)

### Non-Patent Literature

(Non-Patent literature 1) Chem. Commun., 2018, 54, pp.6184-6187

### SUMMARY OF THE INVENTION

The present disclosure has been made to solve the above problems. An objective of the present disclosure is to provide a two-pot-two-step method and a system therefor, the method and system simultaneously performing a process of converting hydrocarbon containing at least one hydroxy group and a process of converting carbon dioxide in different pots, thereby increasing the process yield.

In addition, the other objective of the present disclosure is to provide an integrated process and a system therefor, the process and system enabling hydrogen obtained in the process of converting hydrocarbon containing at least one hydroxy group to be directly used in the process of converting carbon dioxide.

In one embodiment of the present disclosure, a two-step simultaneous conversion method for carbon dioxide and a hydrocarbon containing at least one hydroxy group includes: a hydrocarbon conversion step in which water and metal oxalates are added to and reacted with a hydrocarbon containing at least one hydroxy group to produce a metal salt that is a dehydrogenated form of the hydrocarbon, hydrogen, and water; a hydrogen separation step of separating hydrogen from the effluent of the hydrocarbon conversion step; and a carbon dioxide conversion step in which the hydrogen obtained in the hydrogen separation step is reacted with carbon dioxide and/or carbon dioxide-derived carbonate so that the carbon dioxide is converted into formate and water.

In addition, as an embodiment, the hydrogen separation step is characterized in that the hydrogen is separated while a pressure higher than a required pressure for the carbon dioxide conversion step is maintained.

In addition, as an embodiment, the two-step simultaneous conversion method for carbon dioxide and a hydrocarbon containing at least one hydroxy group may further include: a first water separation step of separating predetermined portion of water from the effluent from which hydrogen has been separated and removed in the hydrogen separation step; a first carbon dioxide-derived carbonate production step in which a carbon dioxide-derived carbonate and a compound in which a metal is substituted with an alkyl group in a metal salt that is a dehydrogenated form of the hydrocarbon are produced by adding and reacting carbon dioxide and at least one of the C1 to C5 alcohols to a received effluent after a predetermined portion of water is separated from the first water separation step; and a first carbon dioxide-derived carbonate separation step of separating the carbon dioxide-derived carbonate produced in the first carbon dioxide-derived carbonate producing step, in which the carbon dioxide-derived carbonate separated in the first carbon dioxide-derived carbonate separation step is supplied to the carbon dioxide conversion step and used as a carbon dioxide source.

In addition, as an embodiment, in the two-step simultaneous conversion method for carbon dioxide and a hydrocarbon containing at least one hydroxy group, the method further includes a first alcohol separation step of separating alcohol and a compound in which a metal is substituted with an alkyl group in a metal salt that is a dehydrogenated form of the hydrocarbon by receiving the effluent after the carbon dioxide-derived carbonate is separated from the first carbon dioxide-derived carbonate separation step.

In addition, as an embodiment, in the two-step simultaneous conversion method for carbon dioxide and a hydrocarbon containing at least one hydroxy group, the method further includes a second water separation step of separating water from the effluent of the carbon dioxide conversion step.

In addition, as another embodiment, the second water separation step may be configured to separate predetermined portion of water rather than completely separate water from the effluent of the carbon dioxide conversion step. In this case, in the two-step simultaneous conversion method for carbon dioxide and a hydrocarbon containing at least one hydroxy group, the method further includes: a second water separation step of separating a portion of water from the effluent of the carbon dioxide conversion step; a second carbon dioxide-derived carbonate production step in which alkyl formate and carbon dioxide-derived carbonate are produced by adding and reacting at least one of C1 to C5 alcohols, and carbon dioxide to a received effluent after a predetermined portion of water is separated from the second water separation step; a second carbon dioxide-derived carbonate separation step of separating the carbonate produced in the second carbon dioxide-derived carbonate producing step, and the carbon dioxide-derived carbonate separated in the second carbon dioxide-derived carbonate separation step is returned to the carbon dioxide conversion step and used as a carbon dioxide source.

In addition, as an embodiment, in the two-step simultaneous conversion method for carbon dioxide and a hydrocarbon containing at least one hydroxy group, the method further includes a second alcohol separation step of separating alcohol and alkyl formate by receiving the effluent after the carbon dioxide-derived carbonate is separated from the second carbon dioxide-derived carbonate separation step.

In addition, as an embodiment, the second alcohol separation step may include: a 2-1 separation step of separating alcohol and other substances by receiving the effluent after the carbon dioxide-derived carbonate is separated from the second carbon dioxide-derived carbonate separation step; and a 2-2 separation step of separating the alkyl formate and water from the material from which the alcohol was separated in the 2-1 separation step.

In addition, as an embodiment, the hydrocarbon containing at least one hydroxy group is one or a mixture selected from glucose; maltose; galactose; xylose; sorbitol; mannitol; galactitol; xylitol; glycerol; 1,4-butanediol or an isomer thereof; 1,4-pentanediol or an isomer thereof; 1,2-propanediol or an isomer thereof; butanol; pentanol; propanol; chitin-derived compounds.

In addition, in the two-step simultaneous conversion system for a hydrocarbon containing at least one hydroxyl group and carbon dioxide according to another embodiment of the present disclosure, the two-step simultaneous conversion system includes: a 1-1 reactor used in the process of converting hydrocarbons containing at least one hydroxy group according to the present disclosure into a metal salt that is a dehydrogenated form of the hydrocarbon, hydrogen, and a product including water in the presence of water and metal; a hydrogen separator for separating hydrogen from the effluent of the 1-1 reactor; and a 1-2 reactor that receives hydrogen from the hydrogen separator and converts it to formate and water by reacting the received hydrogen with carbon dioxide-derived carbonate.

In addition, as an embodiment, in the two-step simultaneous conversion system for carbon dioxide and a hydrocarbon containing at least one hydroxy group, according to another embodiment of the present disclosure, the two-step simultaneous conversion system includes: a first water separator for separating a predetermined portion of water by receiving the effluent after hydrogen is separated from the hydrogen separator; a 2-1 reactor for producing a compound in which a metal is substituted with an alkyl group in a metal salt that is a dehydrogenated form of the hydrocarbon and carbon dioxide-derived carbonate by introducing and reacting carbon dioxide with any one of C1 to C5 alcohols after receiving the effluent being removed predetermined portion of water from the first water separator; a first carbonate separator for separating carbon dioxide-derived carbonates produced in the 2-1 reactor; a first product separator for separating alcohol and a compound in which the metal is substituted with an alkyl group in a metal salt that is a dehydrogenated form of the hydrocarbon by receiving an effluent after carbon dioxide-derived carbonate is separated from the first carbonate separator; and a second water separator for separating a predetermined portion of water by receiving the effluent from the 1-2 reactor, and carbon dioxide-derived carbonate separated from the first carbonate separator is supplied to the 1-2 reactor and used as a carbon dioxide source.

In addition, as another embodiment, in the two-step simultaneous conversion system for carbon dioxide and a hydrocarbon containing at least one hydroxy group, according to another embodiment of the present disclosure, the two-step simultaneous conversion system includes: a first water separator for separating a predetermined portion of water by receiving the effluent after hydrogen is separated from the hydrogen separator; a 2-1 reactor for producing a compound in which a metal is substituted with an alkyl group in a metal salt that is a dehydrogenated form of the hydrocarbon and carbon dioxide-derived carbonate by introducing and reacting carbon dioxide with any one of C1 to C5 alcohols after receiving the effluent being removed predetermined portion of water from the first water separator; a first carbonate separator for separating carbon dioxide-derived carbonates produced in the 2-1 reactor; a first product separator for separating alcohol and a compound in which a metal is substituted with an alkyl group in a metal salt that is a dehydrogenated form of the hydrocarbon by receiving an effluent after carbon dioxide-derived carbonate is separated from the first carbonate separator; and a second water separator for separating predetermined portion of water by receiving the effluent from the 1-2 reactor; a 2-2 reactor for producing alkyl formate and carbon dioxide-derived carbonate by introducing and reacting carbon dioxide with any one of C1 to C5 alcohols after receiving the effluent after a predetermined portion of water is removed from the second water separator; a second carbonate separator for separating carbon dioxide-derived carbonates produced in the 2-2 reactor; and a second product separator which separates alcohol and alkyl formate by receiving an effluent after the carbon dioxide-derived carbonate is separated from the second carbonate separator, and the carbon dioxide-derived carbonate separated from the first carbonate separator and the second carbonate separator is supplied to the 1-2 reactor and used as a carbon dioxide source.

According to the present disclosure, in the simultaneous conversion reaction for carbon dioxide and a hydrocarbon containing at least one hydroxy group, the conversion process of a hydrocarbon and the conversion process of carbon dioxide are separately performed, thereby setting optimal reaction conditions for each process, thereby increasing the yield of each process and reducing the production of by-products.

In addition, the present disclosure separately performs a conversion process for carbon dioxide and a hydrocarbon containing at least one hydroxy group. Hydrogen obtained through a conversion reaction of a hydrocarbon containing at least one hydroxy group may be immediately used as a raw material for a conversion process of carbon dioxide-derived carbonate, thereby increasing process energy efficiency and convenience.

### BRIEF DESCRIPTION OF THE DRAWINGS

FIG. 1 is a block diagram for explaining a method for the two-step simultaneous conversion system for carbon dioxide and a hydrocarbon containing at least one hydroxy group according to an embodiment of the present disclosure;

FIG. 2 is a block diagram for explaining a two-step simultaneous conversion method for carbon dioxide and a hydrocarbon containing at least one hydroxy group according to another embodiment of the present disclosure;

FIG. 3 is a conceptual diagram for explaining a system for the two-step simultaneous conversion for carbon dioxide and a hydrocarbon containing at least one hydroxy group according to an embodiment of the present disclosure; and

FIG. 4 is a block diagram for explaining a two-step simultaneous conversion method of glycerol as a hydrocarbon, including at least one hydroxy group and carbon dioxide, according to an embodiment of the present disclosure.

### DESCRIPTION OF THE PREFERRED EMBODIMENTS

Hereinafter, a two-step simultaneous conversion system for carbon dioxide and a hydrocarbon containing at least one hydroxy group and a method thereof will be described in detail with reference to the accompanying drawings so that those skilled in the art may easily implement the present disclosure.

In the detailed description of the principle of the preferred embodiment of the present disclosure, if it is determined that a detailed description of a related known function or configuration may unnecessarily obscure the gist of the present disclosure, the detailed description thereof will be omitted.

In addition, since the configurations shown in the drawings and embodiments described herein are only the most preferred embodiments of the present disclosure and do not represent all the technical spirit of the present disclosure, it should be understood that there may be various equivalents and modified examples that may replace them at the time of the present application.

The present disclosure relates to a method and system for simultaneous conversion for carbon dioxide and a hydrocarbon containing at least one hydroxy group through a catalytic reaction. The present disclosure includes a two-pot-two-step process in which hydrocarbon containing at least one hydroxy group is dehydrogenated to produce hydrogen, and the hydrogen is immediately introduced into a carbon dioxide conversion process to convert carbon dioxide.

In the present disclosure, the “hydrocarbon containing at least one hydroxy group” may be one selected from monohydric alcohol or polyhydric alcohol or a mixture thereof, and the hydrocarbon containing at least one hydroxy groups preferable in terms of reactivity with carbon dioxide to be one selected from the group consisting of glucose; maltose; galactose; xylose; sorbitol; mannitol; galactitol; xylitol; glycerol; 1,4-butanediol or an isomer thereof; 1,4-pentanediol or an isomer thereof; 1,2-propanediol or an isomer thereof; butanol, pentanol and propanol; and chitin-derived compounds, or a mixture thereof. Glucose, xylose, and glycerol are more preferable, including an environmental perspective and a raw material supply and demand perspective.

Hereinafter, the two-step simultaneous conversion method for carbon dioxide and a hydrocarbon containing at least one hydroxy group according to the present disclosure will be described in detail.

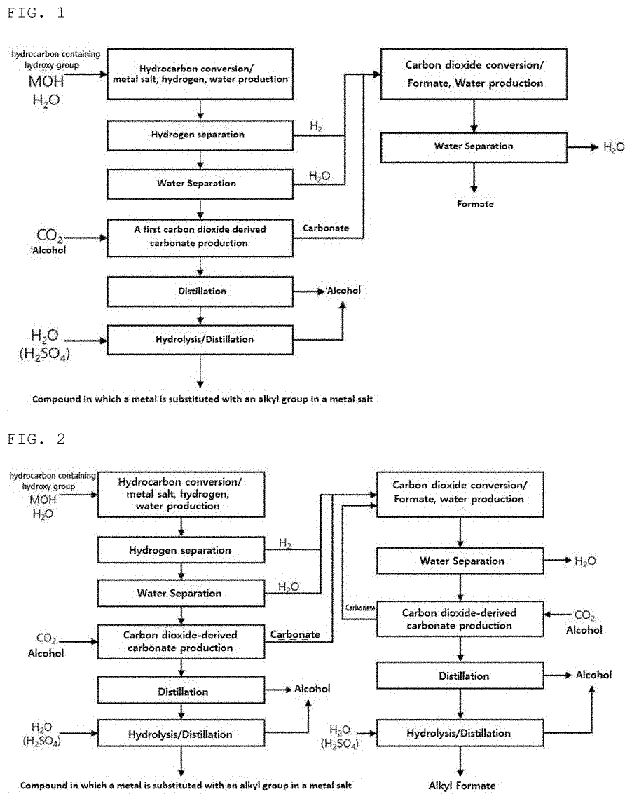

FIG. 1 is a block diagram for explaining a method for the two-step simultaneous conversion system for carbon dioxide and a hydrocarbon containing at least one hydroxy group according to an embodiment of the present disclosure.

As shown in FIG. 1, the present disclosure provides a two-step simultaneous conversion method for carbon dioxide and a hydrocarbon containing at least one hydroxy group, the method includes: a hydrocarbon conversion step in which water and metal oxalates are added and reacted to the hydrocarbon containing at least one hydroxy groups to produce a metal salt that is a dehydrogenated form of the hydrocarbon, hydrogen, and water; the hydrogen separation step of separating hydrogen from the effluent of the hydrocarbon conversion step; a carbon dioxide conversion step of receiving hydrogen from the hydrogen separation step and converting it into formate and water by reacting the received hydrogen with carbon dioxide and/or carbon dioxide-derived carbonate.

In the hydrocarbon conversion step, a hydrocarbon containing at least one hydroxy group is reacted with a metal oxalate in the presence of water to produce a metal salt that is a dehydrogenated form of the hydrocarbon, hydrogen, and water.

The metal oxalate may be at least one selected from alkali metal oxalates and alkaline earth metal oxalates, preferably alkali metal oxalates, and more preferably NaOH and/or KOH.

The molar ratio of the metal oxalate and the hydrocarbon containing at least one hydroxy group may usually be 1:10 to 10:1. In addition, a molar ratio of water and the hydrocarbon containing at least one hydroxy group may be 1:1 to 2:1.

The reaction in the hydrocarbon conversion step is performed in a temperature range of 50° C. to 300° C. and a pressure range of 1 to 200 bar, preferably in a temperature range of 100° C. to 250° C. and a pressure range of 20 to 120 bar.

The hydrogen separation step is a step of separating hydrogen from the effluent of the hydrocarbon conversion step. The hydrogen separation may be performed by using a general separation device such as a flash drum, a separation membrane, and an adsorption tower, and for process efficiency, hydrogen is separated at a pressure greater than or equal to a pressure required in the carbon dioxide conversion step, so the pressure of the separated hydrogen may be greater than or equal to the pressure required in the carbon dioxide conversion step.

By doing so, the hydrogen separated in the hydrogen separation step may be directly used for the carbon dioxide conversion reaction without a separate pressurization treatment, and a separation device for maintaining the pressure of the carbon dioxide conversion reaction may be omitted so that the process efficiency may be improved. Typically, the pressure of hydrogen after separation obtained in the hydrogen separation step may be 20 to 120 bar.

The carbon dioxide conversion step is a step of producing formate by reacting carbon dioxide-derived carbonate with the hydrogen obtained in the hydrogen separation step in the presence of water.

The “carbon dioxide-derived carbonate” means a metal carbonate or metal bicarbonate produced by reacting a metal and carbon dioxide, and the metal may preferably be at least one selected from alkali metal and alkaline earth metal. In addition, the “carbon dioxide-derived carbonate” may preferably be a carbonate and/or bicarbonate of alkali metal, more preferably at least one of potassium bicarbonate (KHCO₃), sodium bicarbonate (NaHCO₃), and cesium bicarbonate (CsHCO₃), and most preferably, it is potassium bicarbonate (KHCO₃).

As described above, by using the alkali carbonate as the carbon dioxide source, the carbonate produced from the carbon dioxide-derived carbonate may be recovered and used again in connection with the carbon dioxide-derived carbonate production step described later rather than when carbon dioxide itself is used as a reactant. In addition, the present disclosure further includes: a first water separation step of separating a predetermined portion of water from the effluent after hydrogen has been separated and removed in the hydrogen separation step; a first carbon dioxide-derived carbonate production step in which a carbon dioxide-derived carbonate and a compound in which a metal is substituted with an alkyl group in a metal salt that is a dehydrogenated form of the hydrocarbon are produced by receiving an effluent after predetermined portion of water is separated from the first water separation step and by adding and reacting carbon dioxide with alcohols; and a first carbon dioxide-derived carbonate separation step of separating the carbon dioxide-derived carbonate produced in the first carbon dioxide-derived carbonate producing step. The alcohol may preferably be a C1 to C5 alcohol, for example, methanol, ethanol, n-propanol, i-propanol, n-butanol, i-butanol, t-butanol, n-pentanol, i-pentanol.

The first water separation step is a step of separating a predetermined portion of water from the effluent after hydrogen has been separated and removed in the hydrogen separation step. In this case, the purpose is to remove some water. Water is separated and removed so that the mass ratio of the amount of water remaining after some water is separated and removed and the metal salt that is a dehydrogenated form of the hydrocarbon containing at least one hydroxy group is 0.1% to 15%. By controlling the content of water in advance, in the step of producing a carbon dioxide-derived carbonate to be described below, the production yield of the carbon dioxide-derived carbonate may be increased. In addition, the water separated in the first water separation step may be used in the carbon dioxide conversion step. Thereafter, the first carbon dioxide-derived carbonate production step is a step of substituting a metal with an alkyl group and carbonating the substituted metal in a metal salt that is a dehydrogenated form of the hydrocarbon by adding carbon dioxide and alcohol to the effluent after predetermined portion of water is separated in the first water separation step. In this case, the alcohol may preferably be a C1 to C5 alcohol.

The number of moles of the added alcohol may be 1 to 100 times greater than the number of moles of the metal salt that is a dehydrogenated form of the hydrocarbon containing at least one hydroxy group.

The carbon dioxide gas may be supplied under pressure in the range of 1 to 200 bar, and the temperature at this time may be in the range of room temperature to 300° C., preferably in the range of 10 to 50 bar, room temperature to 200° C.

Thereafter, the first carbon dioxide-derived carbonate separation step is a step of separating the produced carbon dioxide-derived carbonate. The produced metal carbonate is precipitated in a solution phase in the form of crystals, and the precipitated metal carbonate can be separated through filtration, centrifugation, or filter methods.

The separated metal carbonate may be supplied to the carbon dioxide conversion step and recycled as a carbon dioxide source.

In addition, the present disclosure may further include a first alcohol separation step of receiving an effluent after the carbon dioxide-derived carbonate is separated from the first carbon dioxide-derived carbonate separation step to separate alcohol and a compound in which a metal is substituted with an alkyl group in a metal salt that is a dehydrogenated form of the hydrocarbon.

The first alcohol separation step may further include: a step of primarily separating alcohol through distillation by receiving an effluent after the carbon dioxide-derived carbonate is separated from the first carbon dioxide-derived carbonate separation step; and a step of secondarily separating alcohol through hydrolysis and distillation by receiving water or sulfuric acid to the effluent from which the alcohol is primarily separated. Since these separation steps can be separated using a method conventional in the art to which the present disclosure pertains, a detailed description is omitted.

On the other hand, a second water separation step of removing water from the effluent discharged in the carbon dioxide conversion step may be performed. In FIG. 1, since the metal formate itself produced in the carbon dioxide conversion step is the target product, in the second water separation step, the water may be completely separated from the effluent after the carbon dioxide conversion step to obtain a pure metal formate as a product.

FIG. 2 is a block diagram for explaining a two-step simultaneous conversion method for carbon dioxide and a hydrocarbon containing at least one hydroxy group according to another embodiment of the present disclosure.

As shown in FIG. 2, in addition to the two-step simultaneous conversion method for carbon dioxide and a hydrocarbon containing at least one hydroxy group according to FIG. 1, the present disclosure further includes: a second water separation step of separating a predetermined portion of water from the effluent of the carbon dioxide conversion step; a second carbon dioxide-derived carbonate production step of producing an alkyl formate in which a metal is substituted with an alkyl group in the formate by adding and reacting carbon dioxide with any one of C1 to C5 alcohols after receiving the effluent being removed predetermined portion of water from the second water separation step; and a second carbon dioxide-derived carbonate separation step of separating the carbonate produced in the second carbon dioxide-derived carbonate production step.

In this case, in the second water separation step, water is separated and removed so that the amount of water remaining after separation and removal is 0.1 to 15% by mass relative to the formate. By controlling the content of water in advance, in the second carbon dioxide-derived carbonate production step, the production yield of carbon dioxide-derived carbonate may be increased.

Since the second carbon dioxide-derived carbonate producing step and the second carbon dioxide-derived carbonate separating step is performed under similar conditions to the above-described first carbon dioxide-derived carbonate producing step and first carbon dioxide-derived carbonate separating step, the same technique will be omitted.

In addition, the present disclosure may further include a second alcohol separation step of separating the alcohol and the alkyl formate by receiving the effluent after the carbon dioxide-derived carbonate is separated from the second carbon dioxide-derived carbonate separation step. Since the second alcohol separation step is performed under similar conditions to the first alcohol separation step, the same technique is omitted. However, the second alcohol separation step may include: a 2-1 separation step of separating the alcohol and the alkyl formate by receiving the effluent after the carbon dioxide-derived carbonate is separated from the second carbon dioxide-derived carbonate separation step; and a 2-2 separation step of separating the alkyl formate and water from the substances from which the alcohol was separated in the 2-1 separation step.

The present disclosure provides a two-step simultaneous conversion system for a carbon dioxide and a hydrocarbon containing at least one hydroxy group.

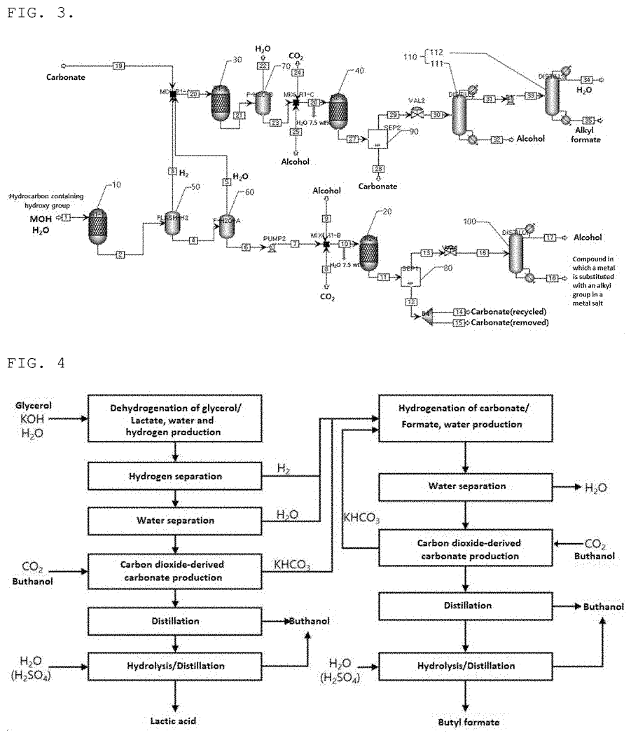

FIG. 3 is a conceptual diagram for explaining a system for the two-step simultaneous conversion for carbon dioxide and a hydrocarbon containing at least one hydroxy group according to an embodiment of the present disclosure.

As shown in FIG. 3, a two-step simultaneous conversion system configured to including: a 1-1 reactor used in the process of converting hydrocarbons containing at least one hydroxy group according to the present disclosure into a metal salt that is a dehydrogenated form of the hydrocarbon, hydrogen, and a product including water in the presence of water and metal oxalate; a hydrogen separator for separating hydrogen from the effluent of the 1-1 reactor; and a 1-2 reactor in which hydrogen supplied from the hydrogen separator reacts with carbon dioxide and/or carbon dioxide-derived carbonates to convert them into formate and water.

In another embodiment of the present disclosure, in the system of this disclosure, a first water separator for separating a predetermined portion of water by receiving an effluent after hydrogen is separated from the hydrogen separator may be positioned at the rear end of the hydrogen separator. A 2-1 reactor for producing a compound in which a metal is substituted with an alkyl group in a metal salt that is a dehydrogenated form of the hydrocarbon and carbon dioxide-derived carbonate by introducing and reacting carbon dioxide with any one of C1 to C5 alcohols after receiving the effluent being removed predetermined portion of water from the first water separator may be positioned at the rear end of the first water separator. A first carbonate separator for separating the carbon dioxide-derived carbonate produced in the 2-1 reactor may be present at the rear end of the 2-1 reactor. In this case, the carbon dioxide-derived carbonate separated in the first carbonate separator may be supplied to the 1-2 reactor and used as a carbon dioxide source.

In addition, the first product separator for separating a compound in which a metal is substituted with an alkyl group in a metal salt that is a dehydrogenated form of the hydrocarbon and alcohol by receiving an effluent after the carbonate is separated from the first carbonate separator may be present at the rear end of the first carbonate separator.

In addition, a second water separator for receiving the effluent from the 1-2 reactor to separate water is included. It is possible to obtain a metal formate in a form in which moisture is completely removed through the second water separator.

Also, in another embodiment of the present disclosure, the system of the present disclosure may further include a process of converting the metal formate produced in the 1-2 reactor to an alkyl formate. In this case, in the system of the present disclosure, the second water separator receives the effluent from the 1-2 reactor and separates a predetermined portion of water rather than completely removing water.

In addition, a 2-2 reactor for producing alkyl formate and carbon dioxide-derived carbonate by introducing and reacting carbon dioxide and any one of C1 to C5 alcohol after receiving the effluent after a predetermined portion of water is removed from the second water separator may be positioned at the rear end of the second water separator, and a second carbonate separator for separating the carbon dioxide-derived carbonate produced in the 2-2 reactor may be present at a rear end of the 2-2 reactor. In this case, the carbon dioxide-derived carbonate separated from the second carbonate separator may be supplied to the 1-2 reactor and used as a carbon dioxide source, as the carbon dioxide-derived carbonate separated from the first carbonate separator.

In addition, at the rear end of the second carbonate separator, a second product separator for separating alcohol and alkyl formate by receiving an effluent after carbon dioxide-derived carbonate is separated in the second carbonate separator may be present.

In addition, in the second product separator, a 2-1 product separator for separating alcohol from other products by receiving the effluent after the carbonate is separated; and a 2-2 product separator that receives the effluent from which alcohol has been separated and removed from the 2-1 product separator and separates the alcohol into the alkyl formate and water may be continuously positioned.

Since the separators can use a conventional separator in the art to which the present disclosure pertains, a detailed description thereof will be omitted. For example, the carbonate separator may use a centrifugal force separator, a filter, and the like, and the product separator may use a distillation device but is not limited thereto.

FIG. 4 is a block diagram for explaining a two-step simultaneous conversion method of glycerol as a hydrocarbon, including at least one hydroxy group and carbon dioxide, according to an embodiment of the present disclosure.

As shown in FIG. 4, the two-step simultaneous conversion method for glycerol and carbon dioxide includes a glycerol conversion step as a process for converting glycerol to lactate, a hydrogen separation step, and a carbon dioxide conversion step as a process for converting carbon dioxide.

As a process for converting the glycerol to lactate, the glycerol conversion step is as follows.

In the glycerol conversion step, as shown in Formula 1 below, water and KOH as a metal oxalate are added to glycerol and reacted to convert potassium lactate, hydrogen, and water.

Glycerol+K⁺+OH⁻→Potassium Lactate+H₂+H₂O [Formula 1]

The reaction can be performed in the presence of a catalyst, and the catalyst may include a catalyst composite in which precious metal is supported as an active metal on a support containing a graphite carbon body, a catalyst in which an organic metal is encapsulated in MOF, a composite metal catalyst including at least one precious metal as the active metal, and at least one selected from another precious metal or non-precious metal is supported on the support. (For example, Pt/Carbon, PtM/Carbon, Pt/ZrO₂, PtM/ZrO₂, M=Ru, Sn, Ni, Co, Rh, Re, etc.)

After the hydrogen separation step, the effluent from which hydrogen is separated and removed is converted into butyl lactate and potassium bicarbonate by adding and reacting carbon dioxide and butanol, as shown in Formula 2 below after some water is removed. At this time, as reaction conditions, the reaction is performed in a temperature range of room temperature to 200° C. and a pressure range of 1 to 100 bar.

Potassium Lactate+CO₂+Butanol →Butyl Lactate+KHCO₃ [Formula 2]

The reaction may be performed without using a catalyst but may also be performed using an acid catalyst such as sulfuric acid.

Meanwhile, a reaction for converting potassium bicarbonate as a carbonate derived from carbon dioxide into formate using hydrogen produced in the conversion reaction of glycerol is as follows.

In the metal carbonate conversion step, as shown in Formula 3 below, water and hydrogen are added to potassium bicarbonate and reacted to convert potassium formate and water. As the reaction conditions of the reaction, the reaction is performed in a temperature range of room temperature to 200° C. and a pressure range of normal pressure to 200 bar.

KHCO₃+H₂→Potassium Formate+Water [Formula 3]

In this case, hydrogen and water used to convert the potassium bicarbonate into potassium formate in the potassium bicarbonate conversion step use hydrogen and water produced when glycerol is converted into potassium lactate in the glycerol conversion step.

To this end, the present disclosure includes: evaporating hydrogen among the reaction products in the glycerol conversion step and supplying hydrogen as a hydrogen source in the potassium bicarbonate conversion step; and evaporating water in the reaction product in the glycerol conversion step, and supplying the water as a water source into the potassium bicarbonate conversion step.

In addition, after the potassium bicarbonate conversion step, the second carbon dioxide-derived carbonate production step may be further performed.

In addition, as the potassium bicarbonate used to convert bicarbonate to formate in the potassium bicarbonate conversion step, potassium bicarbonate produced after the reaction in the first carbon dioxide-derived carbonate production step and the second carbon dioxide-derived carbonate production step may be used.

To this end, the present disclosure includes: a step of separating bicarbonate from the reaction product in the first carbon dioxide-derived carbonate production step, and then supplying the separated bicarbonate as a bicarbonate source in the bicarbonate conversion step; and a step of separating bicarbonate from the reaction product in the second carbon dioxide-derived carbonate production step and then supplying the separated bicarbonate to a bicarbonate source in the bicarbonate conversion step.

In the second carbon dioxide-derived carbonate production step, carbon dioxide and butanol are added and reacted to the formate produced in the bicarbonate conversion step to convert to butyl formate and potassium bicarbonate, as shown in Formula 4 below. At this time, as reaction conditions, the reaction is performed in a temperature range of room temperature to 200° C. and a pressure range of normal pressure to 100 bar.

Potassium formate+CO₂+Butanol →Butyl formate+KHCO₃ [Formula 4]

For reference, in the above schemes, KOH as the base used in the glycerol conversion step and KHCO₃ as the bicarbonate used in the bicarbonate conversion step was described, but in the present disclosure, NaOH may be used as the base used in the glycerol conversion step, NaHCO₃ may be used as the bicarbonate used in the bicarbonate conversion step.

In addition, the two-step simultaneous conversion method of glycerol as a hydrocarbon containing at least one hydroxy group and a carbon dioxide, according to an embodiment of the present disclosure, may include separating lactate and formate from reaction products in the first carbon dioxide-derived carbonate production step and the second carbon dioxide-derived carbonate production step.

More specifically, in the two-step simultaneous conversion method of glycerol and carbon dioxide, according to an embodiment of the present disclosure, the two-step simultaneous conversion method includes: a first distillation step of separating butyl lactate and butanol from the reaction product in the first carbon dioxide-derived carbonate production step from which bicarbonate is separated; a second distillation step of separating butanol from the reaction product in the second carbon dioxide-derived carbonate production step from which bicarbonate is separated; and a third distillation step of separating butyl formate and water from the reaction product in the second carbon dioxide conversion step in which butanol is separated through the second distillation step.

As described above, the present disclosure has been described with reference to the accompanying drawings, which are merely exemplary, and those of ordinary skilled in the art will understand that various modifications and equivalent other embodiments are possible therefrom. Accordingly, the technical protection scope of the present disclosure should be defined by the following claims.

What is claimed is:

1. A two-step simultaneous conversion method for carbon dioxide and a hydrocarbon containing at least one hydroxy group, the method comprising:
a hydrocarbon conversion step of producing a metal salt that is a dehydrogenated form of the hydrocarbon, hydrogen, and water by adding water and a metal oxalate to and reacting with a hydrocarbon containing at least one hydroxy group;
a hydrogen separation step of separating the hydrogen from the effluent of the hydrocarbon conversion step;
a carbon dioxide conversion step of converting carbon dioxide into formate and water by receiving the hydrogen from the hydrogen separation step and reacting the received hydrogen with carbon dioxide and/or carbon dioxide-derived carbonate.

2. The method of claim 1, wherein in the hydrogen separation step, the hydrogen is separated while a pressure equal to or higher than a required pressure for the carbon dioxide conversion step is maintained.

3. The method of claim 1, further comprising:
a first alcohol separation step of separating alcohol and a compound in which a metal is substituted with an alkyl group in a metal salt that is a dehydrogenated form of the hydrocarbon by receiving an effluent after carbon dioxide-derived carbonate is separated from the first carbon dioxide-derived carbonate separation;
a first carbon dioxide-derived carbonate production step in which alcohol and carbon dioxide are added to the effluent after a predetermined portion of the water has been removed from the first water separation step so that a reaction occurs, thereby producing a carbon dioxide-derived carbonate and a compound in which a metal is substituted with an alkyl group in a metal salt that is a dehydrogenated form of the hydrocarbon; and
a first carbon dioxide-derived carbonate separation step of separating the carbon dioxide-derived carbonate produced in the first carbon dioxide-derived carbonate production step,
and the carbon dioxide-derived carbonate separated in the first carbon dioxide-derived carbonate separation step is supplied to the carbon dioxide conversion step and used as a carbon dioxide source.

4. The method of claim 3, further comprising a first alcohol separation step of separating alcohol and a compound from the effluent after carbon dioxide-derived carbonate is separated from the first carbon dioxide-derived carbonate separation step, the compound in which a metal is substituted with an alkyl group in a metal salt that is a dehydrogenated form of the hydrocarbon.

5. The method of claim 4, further comprising a second water separation step of completely separating the water from the effluent of the carbon dioxide conversion step.

6. The method of claim 4, further comprising:
a second water separation step of separating a predetermined portion of water from the effluent of the carbon dioxide conversion step;
a second carbon dioxide-derived carbonate production step in which alkyl formate and carbon dioxide-derived carbonate are produced by adding alcohol and carbon dioxide and reacting the alcohol and the carbon dioxide with the received effluent after a portion of water is separated from the second water separation step; and
a second carbon dioxide-derived carbonate separation step of separating the carbonate produced in the second carbon dioxide-derived carbonate production step,
wherein the carbon dioxide-derived carbonate separated in the second carbon dioxide-derived carbonate separation step is returned to the carbon dioxide conversion step so as to be used as a carbon dioxide source.

7. The method of claim 6, further comprising a second alcohol separation step of separating alcohol and alkyl formate by receiving the effluent after dioxide-derived carbonate is separated from the second carbon dioxide-derived carbonate separation step.

8. The method of claim 1, wherein the hydrocarbon containing at least one hydroxy group is one selected from the group consisting of glucose; maltose; galactose; xylose; sorbitol; mannitol; galactitol; xylitol; glycerol; 1,4-butanediol or an isomer thereof; 1,4-pentanediol or an isomer thereof; 1,2-propanediol or an isomer thereof; butanol; pentanol; propanol; and chitin-derived compounds, or a mixture thereof.

9. The method of claim 7, wherein the second alcohol separation step comprises:
a 2-1 separation step of separating alcohol and other substances by receiving the effluent after the carbon dioxide-derived carbonate is separated from the second carbon dioxide-derived carbonate separation step; and
a 2-2 separation step of separating the alkyl formate and water from the material from which the alcohol was separated in the 2-1 separation step.

10. A two-step simultaneous conversion system for carbon dioxide and a hydrocarbon containing at least one hydroxy group, the system comprising:
a 1-1 reactor that introduces and reacts water and a base to a hydrocarbon containing at least one hydroxy group to convert the hydrocarbon into a metal salt that is a dehydrogenated form of the hydrocarbon, hydrogen, and water;
a hydrogen separator for separating the hydrogen from the effluent of the 1-1 reactor; and
a 1-2 reactor that receives hydrogen from the hydrogen separator and converts the compound to formate and water by reacting with carbon dioxide-derived carbonate.

11. The system of claim 10, comprising:
a first water separator configured to receive the effluent from the hydrogen separator after the hydrogen is separated and to separate a predetermined portion of water;
a 2-1 reactor configured to produce carbon dioxide-derived carbonate and a compound in which a metal is substituted with an alkyl group in a metal salt that is a dehydrogenated form of the hydrocarbon by introducing carbon dioxide to any one of C1 to C5 alcohols after receiving the effluent from which the predetermined portion of water is removed, from the first water separator;
a first carbonate separator configured to separate the carbon dioxide-derived carbonate produced in the 2-1 reactor;
a first product separator configured to separate alcohol and a compound in which a metal is substituted with an alkyl group in a metal salt that is a dehydrogenated form of the hydrocarbon by receiving the effluent of the first carbonate separator after the carbon dioxide-derived carbonate is separated; and
a second water separator configured to receive the effluent of the 1-2 reactor and to separate water from the effluent,
wherein the carbon dioxide-derived carbonate separated by the first carbonate separator is supplied to the 1-2 reactor and is used as a carbon dioxide source.

12. The system of claim 10, comprising:
a first water separator configured to receive the effluent of the hydrogen separator after the hydrogen is separated and to separate a predetermined portion of water from the effluent;
a 2-1 reactor configured to produce carbon dioxide-derived carbonate and a compound in which a metal is substituted with an alkyl group in a metal salt that is a dehydrogenated form of the hydrocarbon by introducing carbon dioxide to any one of C1 to C5 alcohols after receiving the effluent from which the predetermined portion of water is removed, from the first water separator;
a first carbonate separator configured to separate the carbon dioxide-derived carbonate produced in the 2-1 reactor;
a first product separator configured to separate alcohol and a compound in which a metal is substituted with an alkyl group in a metal salt that is a dehydrogenated form of the hydrocarbon by receiving the effluent of the first carbonate separator after the carbon dioxide-derived carbonate is separated;
a second water separator configured to receive the effluent of the 1-2 reactor and to separate a predetermined portion of water from the effluent;
a 2-2 reactor configured to produce alkyl formate and carbon dioxide-derived carbonate by introducing carbon dioxide and any one of C1 to C5 alcohols to the received effluent after predetermined portion of water is removed from the second water separator;
a second carbonate separator configured to separate the carbon dioxide-derived carbonate produced in the 2-2 reactor; and
a second product separator configured to separate alcohol and alkyl formate by receiving the effluent of the second carbonate separator after carbon dioxide-derived carbonate is separated,
wherein the carbon dioxide-derived carbonate separated from the first carbonate separator and the second carbonate separator is supplied to the 1-2 reactor and used as a carbon dioxide source.
